(19) United States
(12) Patent Application Publication
(10) Pub. No.: US 2009/0263493 A1
(43) Pub. Date: Oct. 22, 2009

(54) Method for making synthetic slug slime and medical use of synthetic and or actual slug slime

(76) Inventor: Stephen Chelminski, Antrim, NH (US)

Correspondence Address: George W. Dishong Esq.;DISHONG LAW OFFICE, 40 Bryant Road, Jaffrey, NH, 03452, US

(21) Appl. No.: 12/378,334

(22) Filed: Feb. 13, 2009

Related U.S. Application Data
(60) Provisional application No. 61/204,882, filed on Jan. 12, 2009.
(60) Provisional application No. 61/132,417, filed on Jun. 18, 2008.
(60) Provisional application No. 61/125,077, filed on Apr. 22, 2008.

(51) Int. Cl. A61K 35/24

(52) U.S. Cl. 424/537

(57) ABSTRACT

The device hereof is most preferably the use of the slime coating of slugs or a synthetic copy of the slug slime, healing agents such as steroids antibiotics, growth hormones or pain killers can be mixed is with the slime before application to a burned or infected area. The slime thusly becomes a carrier for the medicines to the affected area as an antibiotic or anti fungal agent. Also, the same material can be used to protect and promote the healing of burns, due to its unique properties, of not only being hydroscopic and/or water absorbing, but also being non water soluble, as well as for its anti-infection properties. Another potentially helpful property of this material, is that it forms a protective skin-like membrane when spread over an area of skin and the observed condition was over burned skin. As an alternative to storing slug slime in the natural hydrated form, slug slime may be dehydrated at low temperatures under vacuum, in order to not harm its molecular structure or the organic chemical constituents which are part of it. After drying, the non hydrated slime may be ground into a powder form. From the powder form, it may be mixed with powdered healing agents, and or added to a solution, for reconstitution/re-hydrating with water, or other liquid in varying concentrations for conveniently spraying the solution or mixture on to burns or wounds in the form of a film. The reconstituted liquefied slime may also be sprayed on to a substrate for application as a sheet, or for applying to bandages for storage and later use. Powdered slug slime may be sprinkled directly on to burns or injuries, without first reconstituting it, allowing the substance to re-hydrate itself in place, on the injury or burn, taking water from the burn or wound. Additionally, there are ways to produce slug slime chemistry without the need to have slugs produce it. Also, cells within the slug which produce the slime may be genetically engineered to produce intended types of human cells instead of or within the slime they normally produce. Further, cells within the slug may be engineered to produce human stem cells, to become part of the slugs structure, or to be expressed as a part of or within the slime. If human cells such as skin cells can be manifested within the slime coating of slugs, the slime with the skin cells present may be applied directly to a burned skin surface. It is even possible, that the slime producing cells of the slug can be engineered to produce the same cells of the patient, by using genetic material from the patient. Slugs cells may be genetically engineered to produce human cells of many types, capable of transplanting to appropriate human body sites.

### CROSS REFERENCE TO RELATED APPLICATIONS/CLAIM FOR PRIORITY

This application claims priority, under 35 USC §119(e), to U.S. Provisional Applications Ser. No. 61/204,882 titled: Medical Use of the Slime Coating of Slugs, filed on Jan. 12, 2009. Provisional Applications Ser. No. 61/132,417 titled: Medical Use of the Slime Coating of Slugs, filed on Jun. 18, 2008. Provisional Applications Ser. No. 61/125,077 titled: Medical Use of the Slime Coating of Slugs, filed on Apr. 22, 2008.

### FIELD OF THE INVENTION

The invention hereof very clearly is identifiable at the present time to the use of natural material found on the surface of slugs for the treatment of medical condition in human beings (may be applicable to other living creatures if more deeply investigated). Medicine components may be considered as defining the field of this invention.

The device hereof is most preferably the use of the slime coating of slugs or a synthetic copy of the slug slime, healing agents such as steroids antibiotics, growth hormones or pain killers can be mixed is with the slime before application to a burned or infected area. The slime thusly becomes a carrier for the medicines to the affected area as an antibiotic or anti fungal agent. Also, the same material can be used to protect and promote the healing of burns, due to its unique properties, of not only being hydroscopic and/or water absorbing, but also being non water soluble, as well as for its anti-infection properties. Another potentially helpful property of this material, is that it forms a protective skin-like membrane when spread over an area of skin and the observed condition was over burned skin. As an alternative to storing slug slime in the natural hydrated form, slug slime may be dehydrated at low temperatures under vacuum, in order to not harm its molecular structure or the organic chemical constituents which are part of it. After drying, the non hydrated slime may be ground into a powder form. From the powder form, it may be mixed with powdered healing agents, and or added to a solution, for reconstitution/re-hydrating with water, or other liquid in varying concentrations for conveniently spraying the solution or mixture on to burns or wounds in the form of a film. The reconstituted liquefied slime may also be sprayed on to a substrate for application as a sheet, or for applying to bandages for storage and later use. Powdered slug slime may be sprinkled directly on to burns or injuries, without first reconstituting it, allowing the substance to re-hydrate itself in place, on the injury or burn, taking water from the burn or wound. Additionally, there are ways to produce slug slime chemistry without the need to have slugs produce it. Also, cells within the slug which produce the slime may be genetically engineered to produce intended types of human cells instead of or within the slime they normally produce. Further, cells within the slug may be engineered to produce human stem cells, to become part of the slugs structure, or to be expressed as a part of or within the slime. If human cells such as skin cells can be manifested within the slime coating of slugs, the slime with the skin cells present may be applied directly to a burned skin surface. It is even possible, that the slime producing cells of the slug can be engineered to produce the same cells of the patient, by using genetic material from the patient. Slugs cells may be genetically engineered to produce human cells of many types, capable of transplanting to appropriate human body sites.

### BACKGROUND OF THE INVENTION/DESCRIPTION OF THE PRIOR ART

There are no Patents of which the Applicant and the inventor of the now disclosed invention is knowledgeable and thus none can be noted herein. Applicant is providing herein all of the information and understanding known to exist. Few if any developments have taken place in this field of technology of finding medical use of the natural covering of slugs. The present invention provides many advantages considered significant and valuable by the inventor hereof.

### SUMMARY OF THE INVENTION

The present invention as disclosed herein and to be claimed in this utility patent application is most simply and most generally the following features and elements.

It is basically disclosed and defined substantially as:

The device hereof is most preferably the use of the slime coating of slugs as an antibiotic or anti fungal agent. Also, the same material can be used to protect and promote the healing of burns, due to its unique properties, of not only being hydroscopic the slime may also be defined as “water absorbing”, but also being non water soluble, as well as for its anti-infection properties. Another potentially helpful property of this material, is that it forms a protective skin-like membrane when spread over an area of burned skin. Slug slime may be dehydrated at low temperatures under vacuum, in order to not harm its molecular structure or the organic chemical constituents which are part of it. After drying, the non hydrated slime may be ground into a powder form for subsequent use thereof. Yet another feature is, that whether it is the natural or a synthetic copy of the slug slime, healing agents such as steroids antibiotics, growth hormones or pain killers can be mixed in with the slime before application to a burned or infected area. The slime thusly becomes a carrier for the medicines to the affected area. Also, cells within the slug which produce the slime may be genetically engineered to produce intended types of human cells instead of or within the slime they normally produce. Further, cells within the slug may be engineered to produce human stem cells, to become part of the slugs structure, or to be expressed as a part of or within the slime. If human cells such as skin cells can be manifested within the slime coating of slugs, the slime with the skin cells present may be applied directly to a burned skin surface. It is even possible, that the slime producing cells of the slug can be engineered to produce the same cells of the patient, by using genetic material from the patient. Slugs cells may be genetically engineered to produce human cells of many types, capable of transplanting to appropriate human body sites.

The invention disclosed herein is a method for medical using slug slime comprising the steps removing natural physical covering of a slug or a plurality of slugs, the covering of slug being identified as slug-slime. The covering of the slug(s) is stored in a means for controllable access by a potential medical user of the removed or the stored slug-slime. The medical user is most generally being of the form of human being, but could be other forms of warm blooded animals of any type. The slug-slime is then used by applying in a predetermined manner to a physical region of the medical user which region is determined as needing medical attention or processing such as problems consisting of thermal burning, physical damage or wound, chemical damage, internal tissue damage, organ damage resulting in some organ pain or malfunction.

An additional feature of the invention includes the potential of repeated application and use of the slug-slime to the region(s) having problems.

Another feature or characteristic of this invention is providing for a method for creating a synthetic copy of slug slime from the natural covering of slugs by first analyzing the composition of chemical and biotechnological components of slug-slime; and genetically and/or chemically engineering such composition of the slug-slime resulting with a means for production of the synthetic copy of slug slime.

Yet another feature is to disclose a method for medical using a synthetic copy of slug slime which method is similar to the method of using natural form of slug-slime. Repeated use of such synthetic copy of slug slime is again similar in form to the repeated use when using natural slug-slime.

Still yet another feature of the invention is the collection and storage and subsequent use of the natural slug-slime and the synthetic slug slime which material is most intened.

These and further aspects and advantages of the present invention will become apparent to those skilled in the art to which this invention pertains after a study of the present disclosure of the invention including the appended claims.

### BRIEF DESCRIPTION OF THE DRAWINGS

Based upon the fundamental characteristics of this disclosed invention, there are no drawings of any significance that could add to the disclosure explanation or definition of this invention. No drawings are therefore included herewith.

### DESCRIPTION OF THE INVENTION

The present invention since it has been discovered for use with the human structure where solutions to undesireable conditions are desired or needed was used in the manners described as follows by the inventor hereof. The consequences of such use have been determined, for many reasons, to be absolutely unexpected/unanticipated especially since there is no evidence known to the applicant/inventor hereof of such application and/or use of the “slug slime” (or a synthetic copy of the slug-slime coating whereby both the natural and the synthetic form of the material may be identified as slug slime unless special characteristics and features are particular to either the natural form of slug slime or the synthetic form of slug slime) for treatment of human physical problems. Additionally, there are ways to produce slug slime chemistry without the need to have slugs produce it. Also, cells within the slug which produce the slime may be genetically engineered to produce intended types of human cells instead of or within the slime they normally produce. Further, cells within the slug may be engineered to produce human stem cells, to become part of the slugs structure, or to be expressed as a part of or within the slime. If human cells such as skin cells can be manifested within the slime coating of slugs, the slime with the skin cells present may be applied directly to a burned skin surface. It is even possible, that the slime producing cells of the slug can be engineered to produce the same cells of the patient, by using genetic material from the patient. Slugs cells may be genetically engineered to produce human cells of many types, capable of transplanting to appropriate human body sites.

To fully and appropriately disclose and teach this invention—the use of slug slime relative to the human body—the inventor will provide a full representation of personal experiments of the personal use of slug slime upon the inventor's body parts such as skin surface having burns thereon, or bacterial or fungal infections on the skin surface.

The invention of Applicant hereof, is to use of the slime coating of slugs (or a synthetic copy of the slug slime coating) as an antibiotic or anti fungal agent. Also, the same material can be used to protect and promote the healing of burns, due to its unique properties, of not only being hydroscopic, or water absorbing, but also being non water soluble, as well as for its anti-infection properties. Another potentially helpful property of this material, is that it forms, at least upon careful examination on the part of Applicant, a protective skin-like membrane when spread over an area of burned skin. On several occasions, inventor treated a persistant skin rash—(note not a thermal created burn but an irregular surface infected condition of the skin). A particular rash was located in the warm moist area between the legs, caused from or by scratching at the annoyed location. The rash was painfull and would not respond to treatment with antibiotic ointment and was severe enough so as to become infected with what appeared to the applicant to be a secondary infection possibly stafflacoccous.

Applicant actually decided to try the slug slime after observing the slugs in the vegetable garden and reasoning that they would be attacked by bacteria or fungus if it were not for their protective coating of slime. In the view of Applicant, it was truly amazing, to find that the infection went away within eight or ten hours after the slug slime was personally applied to the infected area.

With regard to the healing of burns, to test the personal theory of Applicant that the slime would help heal burns, Applicant very carefully but intentially burned the skin on his arm, with a soldering iron, giving himself a secondary burn over a small area. Applicant then spread some slug slime over the burned area and covered it with a plastic surfaced “bandaid” type bandage. The bandage was left on over the slime covered burn over night and the bandage was taken off in the morning. Much to the amusement and pleasure of Applicant, it was observed or found by Applicant, that the slime had dried to form a protective “skin” over the burn. When the coating was touched by Applicant Applicant experienced no painfull sensation which might happen when touching an un-protected burn.

Another interesting facet of this protective slug slime coating became apparent, when Applicant showered and found that after the shower, the coating was still there and after a while it was again dry and continued to provide the protective barrier. Also, Applicant noticed, that the burn did not sting as one might normally expect, when and after showering with an exposed burn. It is the serious belief of Applicant, that this slug slime coating might well be porous enough to provide a path for the delivery of medications or growth hormones or the like to the burned area without removal of the protective coating and may possibly allow oxygen to come in contact with the burned area while keeping out bacteria. A different species of slug were tried on separate infections, and it was apparent that each worked as well as the other.

Because all species of slug live in the same kind of damp environment, teeming with bacterial and fungal life, Applicant based upon common and ordinary thought processes and reflections, would conclude, that all of them would have similar self protecting anti- infection qualities, however likely different, within the makeup of the slime from one to another. It probably should be biologically and/or chemically investigated to determine if slug slime might have anti-viral qualities, or if slugs could be induced to produce such chemicals and for bacteria as well.

The very unexpected and unobvious observations and results achieved by Applicant in the performance of a “form of experimentation” has certainly created a feeling of a need of Applicant to get patentable invention protection for the invention and also to form and disclose a reason for the performance of additional and formal experiments, if for no other reason but to enhance and support in a formal and understood medical experimentation the results observed by Applicant and reported upon herein. The use of slug slime (or a synthetic copy of the slug slime coating) may be functional relative to stem cell use well into the future. It is only important to note that such advantage may exist now or in the near future

Whether it is the natural or a synthetic copy of the slug slime, healing agents such as steroids antibiotics or growth hormones can be mixed is with the slime before application to a burned or infected area. The slime becoming a carrier for the medicines to the affected area. The very unexpected and unobvious observations and results achieved by Applicant in the performance of a “form of experimentation” has certainly created a feeling of a need of Applicant to get patentable invention protection for the invention and also to form and disclose a reason for the performance of additional and formal experiments, if for no other reason but to enhance and support in a formal and understood medical experimentation the results observed by Applicant and reported upon herein. Additionally Applicant expects that there will be found additional advantages medically to use slug slime in treatment. Further, very likely there will be found ways to produce slug slime chemistry without the need to have slugs produce it and have it taken from them and they would then have to reproduce slime and perhaps be exposed to biological and environmental conditions which could cause some problems for the slugs in there lifestyle. Also, cells within the slug which produce the slime may be genetically engineered to produce intended types of human cells instead of or within the slime they normally produce. Further, cells within the slug may be engineered to produce human stem cells, to become part of the slugs structure, or to be expressed as a part of or within the slime. If human cells such as skin cells can be manifested within the slime coating of slugs, the slime with the skin cells present may be applied directly to a burned skin surface. It is even possible, that the slime producing cells of the slug can be engineered to produce the same cells of the patient, by using genetic material from the patient. Slugs cells may be genetically engineered to produce human cells of many types, capable of transplanting to appropriate human body sites.

As an alternative to storing slug slime in the natural hydrated form, slug slime may be dehydrated at low temperatures under vacuum, in order to not harm its molecular structure or the organic chemical constituents which are part of it. After drying, the non hydrated slime may be ground into a powder form. From the powder form, it may be mixed with powdered healing agents, and or added to a solution, for reconstitution/re-hydrating with water, or other liquid in varying concentrations for conveniently spraying the solution or mixture on to bums or wounds in the form of a film. The reconstituted liquefied slime may also be sprayed on to a substrate for application as a sheet, or for applying to bandages for storage and later use.

Powdered slug slime (or a synthetic copy of the slug-slime coating) may be sprinkled directly on to bums or injuries, without first reconstituting it, allowing the substance to re-hydrate itself in place, on the injury or bum, taking water from the bum or wound. In re-hydrating itself, slug slime would, in some manner and very likely revert back to the form it, held before dehydrating.

What is claimed is:

1. A method for medical using slug slime comprising the steps:
removing natural physical covering of a slug or a plurality of slugs, said covering of slug being identified as slug-slime;
storing said slug-slime removed from said slug or slugs in a means for controllable access by a potential medical user of said removed or said stored slug-slime, said medical user being of the form of human being, warm blooded animals of any type; and
applying in a predetermined manner said slug-slime to a physical region of said medical user determined as needing medical processing selected from the group consisting of thermal burning, physical damage or wound, chemical damage, internal tissue damage, organ damage resulting in some organ pain or malfunction.

2. The method of claim 1 wherein said method for medical using of slug-slime, further comprising the steps:
repeating said method for medical use of slug-slime as determined to be needed for continual and effective medical treatment using said slug-slime.

3. The method of claim 1, including the steps of adding medicinal components to said slug-slime such as pain killers, growth hormones, steroids, anti-bacterial agents, other healing agents or skin cells, before applying to the effected area.

4. A method for creating a synthetic copy of slug slime comprising the steps:
removing natural physical covering of a slug or a plurality of slugs, said covering of slug being identified as slug-slime;
analyzing the composition of chemical and biotechnological components of said slug-slime; and
genetically and or chemically engineering said composition of said slug-slime resulting with a means for production of said synthetic copy of slug slime.

5. A method for medical using a synthetic copy of slug slime comprising the steps:
removing natural physical covering of a slug or a plurality of slugs, said covering of slug being identified as slug-slime;
analyzing the composition of chemical and biotechnological components of said slug-slime;
genetically and or chemically engineering said composition of said slug-slime resulting with a means for production of said synthetic copy of slug slime;
storing said synthetic copy of slug slime in a means for controllable access by a potential medical user of said synthetic slug slime, said medical user being of the form of human being, warm blooded animals of any type; and
applying in a predetermined manner said synthetic slug slime to a physical region of said medical user determined as needing medical processing selected from the group consisting of thermal burning, physical damage or wound, chemical damage, internal tissue damage, organ damage resulting in some organ pain or malfunction.

6. The method of claim 5 wherein said method for medical using a synthetic copy of slug slime, further comprising the steps:
repeating said applying in a predetermined manner said synthetic slug slime to a physical region of said medical user determined as needing medical processing selected from the group consisting of thermal burning, physical damage or wound, chemical damage, internal tissue damage, organ damage resulting in some organ pain or malfunction.

7. The method of claim 5, including the steps of adding medicinal components to said synthetic copy of slug slime such as pain killers, growth hormones, steroids, anti-bacterial agents, other healing agents or skin cells, before applying to the effected area.

8. A material for use in medical treatment of humans and other forms of animals comprising:
natural physical covering of a slug or a plurality of slugs, said covering of slug being identified as slug-slime and said slug-slime being removed from said slugs; and
said slug-slime removed from said slug or slugs storable in a means for controllable access by a potential medical user of said removed or said stored slug-slime, said medical user being of the form of human being, warm blooded animals of any type.

9. The material of claim 8 wherein said slug-slime is a synthetic copy of slug slime created by the method of claim 4:
composition of chemical and biotechnological components of said slug-slime; and
genetically and chemically engineering said composition of said slug-slime resulting with a means for production of said synthetic copy of slug slime.
